(19) United States
(12) Patent Application Publication
(10) Pub. No.: US 2011/0172496 A1
(43) Pub. Date: Jul. 14, 2011

(54) DEVICE FOR TRANSAXILLARY BREAST AUGMENTATION AND ASSOCIATED METHOD

(76) Inventor: Peter J. Capizzi, Huntersville, NC (US)

(21) Appl. No.: 12/980,904

(22) Filed: Dec. 29, 2010

Related U.S. Application Data
(60) Provisional application No. 61/294,627, filed on Jan. 13, 2010.

(51) Int. Cl. A61B 1/32

(52) U.S. Cl. 600/235

(57) ABSTRACT

Embodiments of the present invention provide a medical device and a method for use with a surgical procedure such as a breast augmentation procedure for facilitating the insertion of a breast implant. For example, one embodiment provides a medical device including a U-shaped member comprising a first portion having a first end, a second portion having a second end, and a third portion extending between the first and second portions opposite the first and second ends. The medical device also includes a handle extending from the first end and an angled member extending from the second end at an oblique angle with respect to a longitudinal axis of the second portion. The angled member and at least a portion of the second portion are configured to be placed through an insertion site in a body for facilitating a surgical procedure, such as guiding a breast implant to a desired location.

### CROSS-REFERENCE TO RELATED APPLICATIONS

This application claims priority to U.S. Provisional Application No. 61/294,627, filed Jan. 13, 2010, which is incorporated by reference herein in its entirety.

### BACKGROUND OF THE INVENTION

1) Field of the Invention

The present invention relates to a medical device for use with a surgical procedure and, in particular, to a medical device for use with transaxillary breast augmentation.

2) Description of Related Art

Various techniques can be employed for performing breast augmentation. Typically breast implants are placed beneath each breast, either on top of or beneath the pectoralis major muscle underlying the breast. The breast implant can be inserted using several different incision sites, such as transaxillary (armpit), periumbilical (navel), areola (nipple), and inframammary (crease). The inframammary incision is the most commonly used since it allows the surgeon to work in close proximity to the breast, thus allowing greater control over implant placement, and typically requires the least operative duration. Furthermore, almost all revision surgeries, should they be necessary, can be performed via this incision.

Transaxillary incisions are often preferred because the incision is made in the armpit such that the breasts are left unscarred and the incision is relatively inconspicuous. The transaxillary breast augmentation procedure requires that about a 2 to 2.5 cm incision be made under the armpit. A tunnel is created to the final destination, and a pocket is then defined between the adjacent layers of the tissue or pectoral muscle. Once the pocket has been formed, a breast implant is inserted into the pocket, aligned, and filled with saline. The implant may become twisted or folded during insertion. When this happens, it is necessary for the surgeon to remove the implant and reinsert it until it is correctly aligned.

The transaxillary technique was initially done “blind” or without an endoscope because none existed to facilitate the procedure. Commonly a saline implant would be placed through this blind approach and very few, if any, silicone implants are implanted using this technique. In fact, there was a time that this approach for silicone breast implants was contraindicated because the implant warranty would become void. The endoscope became popularized in the 1990's, which also coincided with the silicone implant moratorium. Saline implants from 1992 until recently were the most common breast implant placed in the United States. With the popularity of the silicone implants today, the use of the endoscope, and the warranty coverage of the implant for the life of the implant, more breast augmentations are performed now than ever. As of 2009, silicone appears to have become the most common breast implant of choice and is now the most common procedure in plastic surgery.

With that said, the procedure and instrumentation using the transaxillary technique has not changed for almost 20 years. Unlike saline implants which require filling with saline, silicone implants cannot be deflated for insertion and must be placed while they are at their full volume. Additionally, the underarm approach is a remote technique. For saline implants, the retractor instrumentation would typically include using devices such as an Army-Navy retractor, Emory endoscopic retractor, Deaver retractor, Harrington retractor, or malleable retractors. None of the aforementioned devices is ideal for insertion for a silicone implant using the transaxillary technique.

Accordingly, it would be advantageous to provide a medical device for use with surgical procedures such as breast augmentation. In addition, it would be advantageous to provide a medical device that allows for easier placement of a breast implant using the transaxillary technique or other remote insertion site. Moreover, it would be advantageous to provide a medical device that is easy to use and more effective for placing a breast implant, such as a silicone implant, using remote insertion sites.

### BRIEF SUMMARY OF THE INVENTION

Embodiments of the present invention may provide improvements over the prior art by, among other things, providing a medical device and method for use with a surgical procedure such as transaxillary breast augmentation. For example, the medical device may include a U-shaped member comprising a first portion having a first end, a second portion having a second end, and a third portion extending between the first and second portions opposite the first and second ends. The medical device also includes a handle extending from the first end and an angled member extending from the second end at an oblique angle with respect to a longitudinal axis of the second portion. For example, in one embodiment, the angled member extends at an angle of about 25-45° with respect to the longitudinal axis of the second portion. The angled member and at least a portion of the second portion are configured to be placed through an insertion site (e.g. a remote insertion site) in a body for facilitating a surgical procedure, such as guiding a breast implant to a desired location. In one embodiment, the U-shaped member, the handle, and the angled member are integrally formed from a single piece of solid material.

According to various aspects, the angled member and the second portion of the U-shaped member have approximately the same width. For instance, the angled member and the second portion may each have a width of about 1-2 inches. In addition, a respective width of the angled member and the second portion of the U-shaped member may progressively increase along their respective lengths. The angled member may include a concave surface, an opposing convex surface, and a thickness extending therebetween. According to one aspect, the angled member has a length less than the second portion, wherein the angled member may have a length of about 0.5-1 inch. In additional variations, the aspect ratio of the width to thickness of the first, second, and third portions may be about 3-8, while the aspect ratio of the width to thickness of the angled member may be about 3-10.

According to additional aspects, the first and third portions of the U-shaped member have approximately the same width (e.g., about 0.5-1 inch) and thickness. A longitudinal axis of the first portion may be non-parallel to the longitudinal axis of the second portion, while a longitudinal axis of the first portion and the longitudinal axis of the second portion may each be straight. Furthermore, the longitudinal axis of the first portion of the U-shaped member may extend at an oblique angle (e.g., about 95-115°) with respect to a longitudinal axis of the third portion, and a longitudinal axis of the first portion of the U-shaped member may extend at an oblique angle with respect to the handle. In another aspect, the second portion of the U-shaped member has a pair of opposing planar surfaces and a thickness extending therebetween. Moreover, U-shaped member may be various sizes and in one embodiment, the first portion of the U-shaped member has a length of about 4-5 inches, the second portion of the U-shaped member has a length of about 2.5-3.5 inches, and the third portion of the U-shaped member has a length of about 4.5-5.5 inches. A sum of interior angles defined between the first and third portions, the second and third portions, and the second portion and the angled member may be less than about 360°.

According to another aspect of the present invention, a medical device for use with a surgical procedure, such as a breast augmentation procedure for facilitating the insertion of a breast implant, is provided. The medical device includes a U-shaped member comprising a first portion coupled to a second portion, wherein the second portion has a longitudinal axis. The medical device also includes a handle extending from the first portion of the U-shaped member and an angled member extending from the second portion at an oblique angle with respect to the longitudinal axis of the second portion and having a length less than the second portion. The angled member and at least a portion of the second portion of the U-shaped member are configured to be placed through an insertion site in a body for facilitating a surgical procedure.

A further aspect of the present invention is directed to a method, such as a method for inserting a breast implant into a body, including the steps of providing a medical device comprising: a U-shaped member including a first portion coupled to a second portion, the second portion having a longitudinal axis; a handle extending from the first portion; and an angled member extending from the second portion at an oblique angle with respect to the longitudinal axis of the second portion. The method further includes forming an incision in the body, such as at a location remote from a desired implant site, and inserting the angled member and at least a portion of the second portion of the U-shaped member within the incision such that the second portion of the U-shaped member extends from the incision to a desired location, such as the desired implant site, and the angled member is positioned at the desired location. Moreover, the method includes performing a surgical procedure while at least a portion of the second portion of the U-shaped member and the angled member are positioned within the body. For example, the surgical procedure may include guiding a breast implant to the desired implant site while at least a portion of the second portion of the U-shaped member and the angled member are positioned within the body.

Various aspects of the method include forming a transaxillary incision or a transabdominal incision. The guiding step may include guiding a silicone breast implant to the desired implant site. In addition, the inserting step may include positioning the angled member such that the angled member defines a pocket at the desired implant site.

The inserting step may additionally include inserting the second portion of the U-shaped member such that the second portion forms a channel between the incision and the desired implant site.

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 illustrates a perspective view of a medical device according to one embodiment of the present invention;

FIG. 2 illustrates an enlarged perspective view of the medical device shown in FIG. 1;

FIG. 3 illustrates another perspective view of the medical device shown in FIG. 1;

FIG. 4 illustrates various perspective views of a medical device according to one embodiment of the present invention;

FIG. 5 illustrates a plan view of a medical device according to one embodiment of the present invention;

FIG. 5a illustrates a cross-sectional view of the handle taken along section E-E;

FIG. 5b illustrates a cross-sectional view of the angled member taken along section A-A;

FIG. 6 illustrates an end view of the medical device shown in FIG. 5;

FIGS. 6a and 6b illustrate cross-sectional views of the handle taken along sections B-B and C-C, respectively;

FIG. 7 is an end view of the medical device shown in FIG. 5;

FIG. 7a is a cross-sectional view of the U-shaped member taken along section D-D;

FIG. 8 illustrates a perspective view of a medical device according to one embodiment of the present invention;

FIG. 9 illustrates a plan view of the medical device shown in FIG. 8;

FIG. 10 illustrates a perspective view of a medical device according to one embodiment of the present invention;

FIG. 11 illustrates a plan view of the medical device shown in FIG. 10; and

FIG. 12 illustrates a medical device for breast augmentation in use according to one embodiment of the present invention.

### DETAILED DESCRIPTION OF THE INVENTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

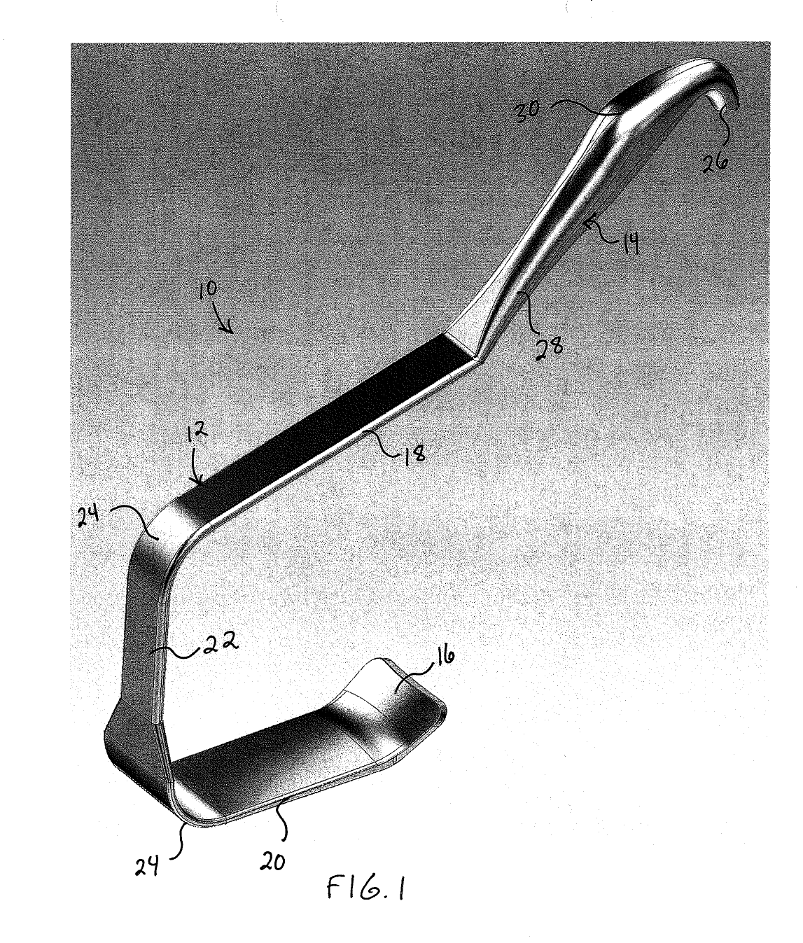

Embodiments of the present invention provide a medical device 10 for use with a breast augmentation procedure and facilitating the insertion of a breast implant. As explained in further detail below and with reference to FIG. 1, a medical device 10 according to one embodiment of the present invention includes a U-shaped member 12, a handle 14 coupled to one end of the U-shaped member, and an angled member 16 coupled to an opposite end of the U-shaped member. The angled member 16 and at least a portion of the U-shaped member 12 are configured to be placed through a remote insertion site for guiding a breast implant to a desired location. The medical device 10 is configured to be used for facilitating placement of a breast implant through a remote insertion site, wherein a remote insertion site may be any site or incision that is spaced away from the breast or desired implant site. For example, the medical device 10 may be employed to deliver a breast implant through a transaxillary or transabdominal incision. In particular, the medical device 10 is configured to deliver silicone breast implants as explained in further detail below, although the medical device may be used for delivering saline or other types of breast implants, including implants of varying size, shape, and texture. In addition, the medical device 10 is configured for use with various surgical or medical procedures beyond breast augmentation. Thus, although the medical device 10 is discussed in the context of breast augmentation, the device may be used with various other surgical or medical procedures for facilitating such a procedure. For instance, the medical device 10 could be used for facilitating placement of an implant or performing a particular surgical procedure at a desired location within the body, wherein a surgical procedure may include one or more steps or acts associated therewith.

Moreover, the U-shaped member 12, the handle 14, and the angled member 16 may be integrally formed from a single piece of solid material. For instance, the medical device 10 may be formed of a variety of metallic and/or non-metallic materials, such as a stainless steel material. However, it is understood that the medical device 10 could be formed of separate components and coupled together if desired. Thus, use of the term “coupled” is not meant to be limiting, as the medical device 10 may be integrally formed or formed from separate components. At least a portion of the U-shaped member 12 and the angled member 16 may comprise a smooth, lubricious surface for facilitating insertion within the body, as well as provide a smooth surface for guiding the implant to the site of implantation.

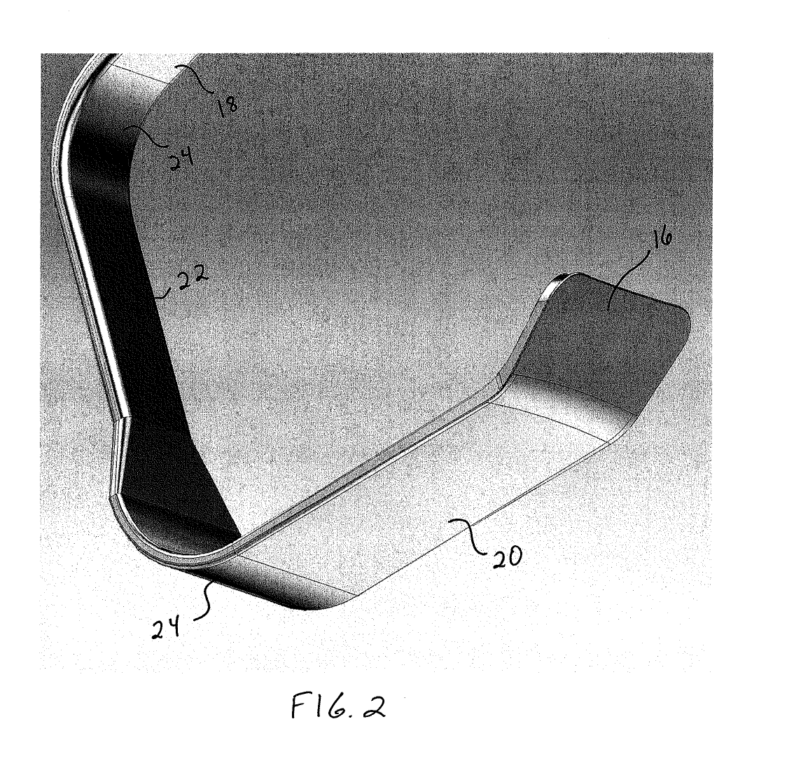

Referring to FIGS. 1-5 and 8-11, a medical device 10 is shown including a U-shaped member 12. The U-shaped member 12 includes a first portion 18, a second portion 20, and a third portion 22 extending between and coupling the first and second portions. Although the term “U-shaped” is used herein, it is understood that the U-shaped member 12 may be generally in the shape of “U”, “C”, or “J”. However, the U-shaped member 12 may include first 18, second 20, and third 22 portions that are of various configurations, sizes, and orientations with respect to one another as will become apparent in the subsequent discussion such that use of the term “U-shaped” is not meant to be limiting.

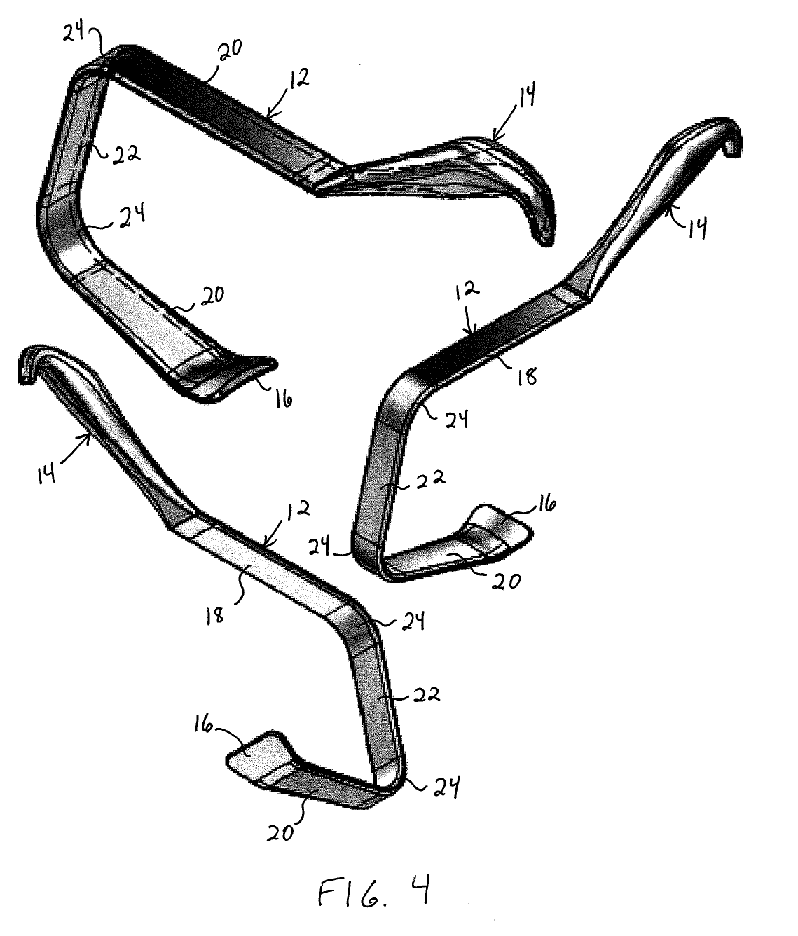

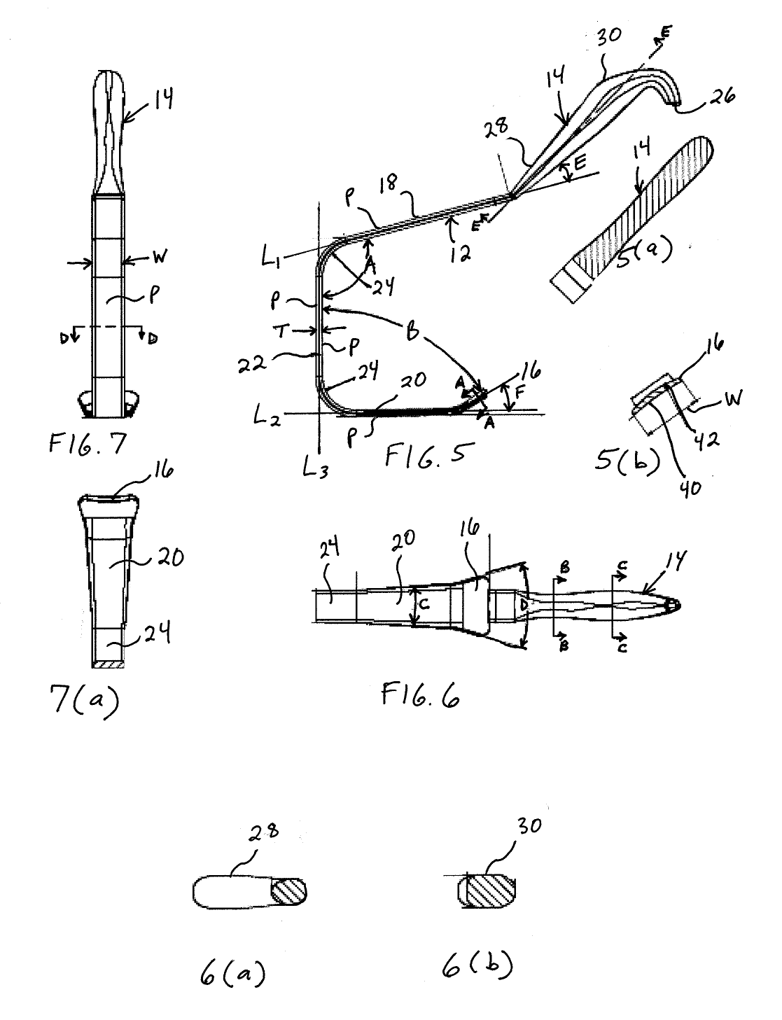

Each of the first 18, second 20, and third 22 portions has a longitudinal axis (L1, L2, and L3, respectively) as shown in FIG. 5. The longitudinal axes L1, L2, and L3 of the first 18, second 20, and third 22 portions, respectively, may be substantially straight and not curved. Thus, the U-shaped member 12 may have three discrete straight portions, although the U-shaped member could include curved portions or be a continuously curving member if desired. The third portion 22 may include a pair of radii 24 joining each of the first 18 and second 20 portions together. For example, the radii 24 may be about 0.5-1.5 inch (e.g., about 0.9 inch) in one embodiment. In addition, the first 18, second 20, and third 22 portions of the U-shaped member 12 may each comprise a pair of opposing planar surfaces “P” and a thickness “T” extending therebetween. Thus, the first 18, second 20, and third 22 portions may have a substantially flat profile with opposing planar and parallel surfaces and according to one aspect, may have a rectangular cross section. The planar surfaces of the second portion 20 may allow the second portion to be readily placed through an incision and to a desired implant location, while also providing the ability to hold open the incision and guide an implant to the desired implant location, as described below.

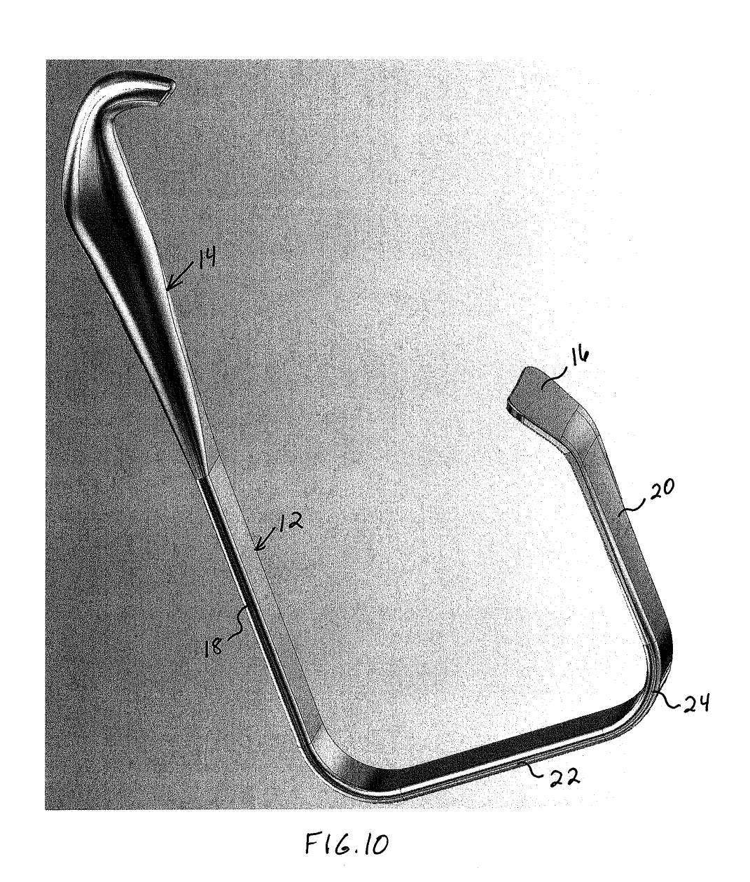

According to one embodiment, the longitudinal axis L1 of the first portion 18 is non-parallel to the longitudinal axis L2 of the second portion 20. However, FIGS. 8-10 illustrate that the longitudinal axes of the first 18 and second 20 portions may be parallel to one another if desired. Moreover, the longitudinal axis of the first portion 18 may extend at an oblique angle with respect to a longitudinal axis of the third portion 22. For example, the longitudinal axis of the first portion 18 may extend at an angle “A” of about 95-115° (e.g., about 105°) with respect to the longitudinal axis of the third portion 22. The oblique angle A defined between the first 18 and third 22 portions may facilitate placement of the U-shaped member 12 with respect to the body, as the oblique angle may allow for the angled member 16 and the second portion 20 to be placed within the incision while also providing space between the handle 14 and the body. The angled member 16 also extends at an oblique angle “F” with respect to the longitudinal axis of the second portion 20. For instance, the angled member 16 may extend at an angle F of about 20-45° (e.g., about 32°) with respect to the longitudinal axis of the second portion 20. Likewise, the angle “B” defined between the longitudinal axis L3 of the third portion 22 and the angled member 16 may be about 45-70° in one embodiment. The angle defined between the angled member 16 and the second portion 20 may facilitate in forming a pocket at the site of implantation and holding the soft tissue up from the pocket, as also described below. A radius may be defined between the end of the second portion 20 and the angled member 16 in order to provide a smooth transition therebetween. Thus, according to one embodiment, a sum of the interior angles defined between the first 18 and third 22 portions, the second 20 and third 22 portions, and the second portion 20 and the angled member 16 is less than about 360°, including less than 345°.

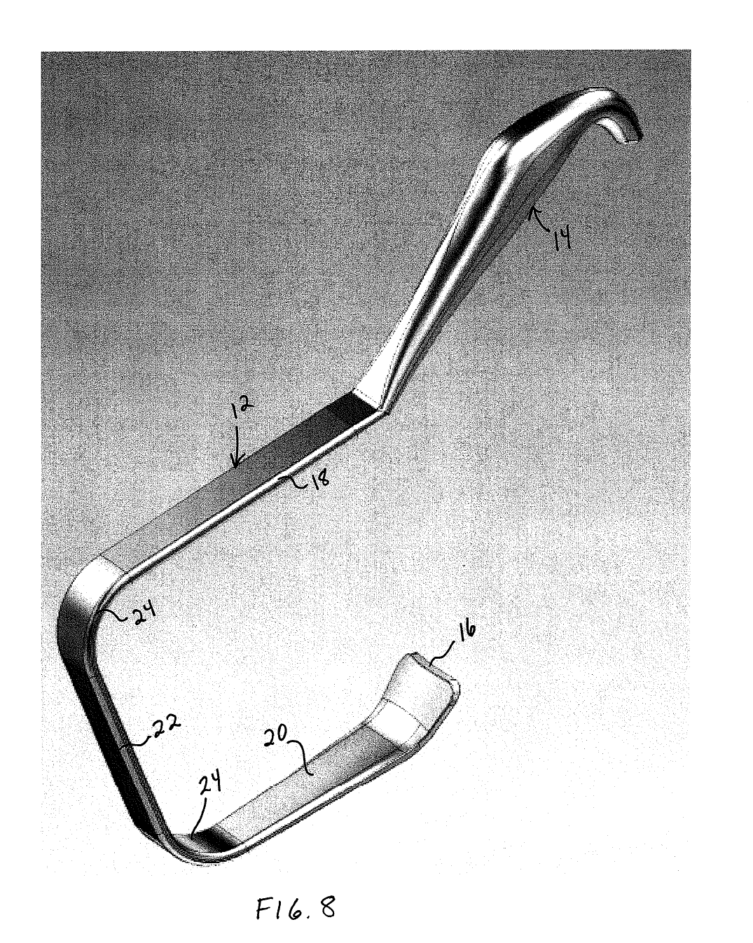

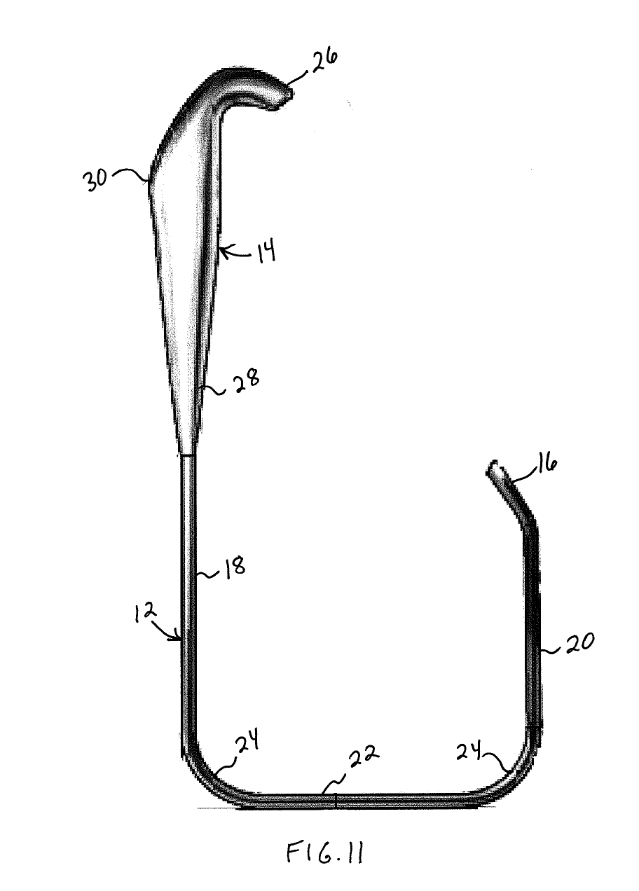

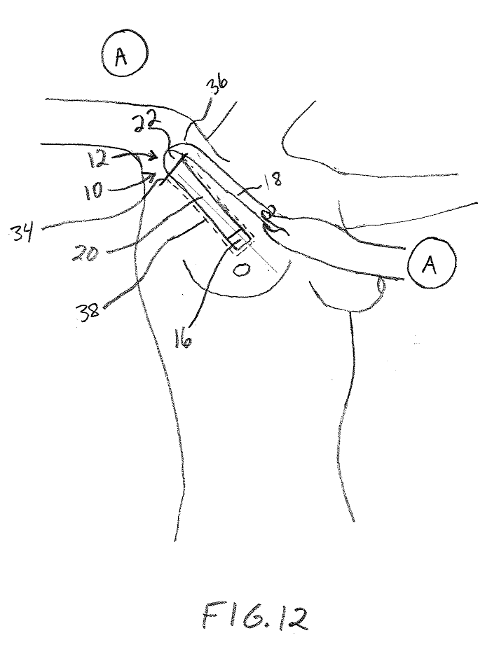

FIGS. 1-6 and 8-11 illustrate that a handle 14 is coupled to the U-shaped member 12. In particular, FIGS. 5, 5a, 6a, and 6b show that the handle 14 shown includes a hook member 26, a tapered portion 28, and a varying cross section along its length, including a raised portion 30. However, the handle 14 may be any suitable size and configuration for allowing an assistant to grip the handle and manipulate the medical device 10 as explained in further detail below. For example, the handle 14 may be about 4.5-5 inches in length (e.g., about 4.9 inches) (see FIG. 5a), while the raised portion 30 has a cross-sectional width of about 0.5-1.5 inches (see FIG. 6b). In use and depending on where an assistant is located with respect to a patient, an assistant would typically grip the handle 14 between the first portion and the hook member 26, wherein the raised portion 30 is positioned within the assistant's palm (see FIG. 12). The longitudinal axis of the first portion 18 may extend at an oblique angle “E” with respect to the handle (e.g., about 30°), as shown in FIGS. 5 and 9. The oblique angle E defined between the first portion 18 and the handle 12 may facilitate placement of the U-shaped member 12 with respect to the body, as the oblique angle E may provide clearance between the patient's body and the handle when in use. However, in an alternative embodiment, the handle 14 may be parallel to the longitudinal axis of the first portion 18, as shown in FIGS. 10 and 11.

According to alternative embodiments, the U-shaped member 12 may be various sizes and configurations. For example, the first 18, second 20, and third 22 portions and the angled member 16 may be various lengths, wherein a length of the third portion 22 is defined along the longitudinal axis L3 between the longitudinal axes L1 and L2 of the first 18 and second portions, the length of the first portion 18 is defined along the longitudinal axis L1 between the end of the radius 24 and the handle 14, and the length of the second portion 20 is defined along the longitudinal axis L2 between the end of the radius 24 and the angled member 16. In one embodiment, the first portion 18 has a length of about 4-5 inches (e.g., about 4.5 inches), the second portion has a length of about 2.5-3.5 inches, the third portion has a length of about 4.5-5.5 inches (e.g., about 4.9 inches), and the angled member has a length of about 0.5-1 inch. The larger length of the third portion 22 provides sufficient spacing between the first 18 and second 20 portions, such as to allow spacing for a patient's armpit while using a transaxillary approach. According to one aspect, the first 18 and third portions 22 may be approximately the same width “W” and thickness “T”, while the second portion 20 and the angled member 16 may have a larger width than the first and third portions (see e.g., FIGS. 1-3, 6 and 7). For instance, the thickness T of the U-shaped member 12 may be about 0.125-0.25 inch (e.g., about 0.2 inches). Furthermore, in one embodiment, the first 18 and third 22 portions have a width W of about 0.5-1 inch (e.g., about 0.9 inches), while the angled member 16 and the second portion 20 each has a width of about 1-2 inches (e.g., about 1.8 inches). In one embodiment, the aspect ratio of the width to thickness of the first 18, second 20, and third 22 portions may be about 3-8 (e.g., about 5), while the aspect ratio of the width to thickness of the angled member 16 may be about 3-10 (e.g., about 8.5). The larger width of the second portion 20 and the angled member 16 aids in keeping an incision open and placing a breast implant, as explained in detail below. For example, the second portion may be sized and configured to hold open an incision of 4.5 cm or greater.

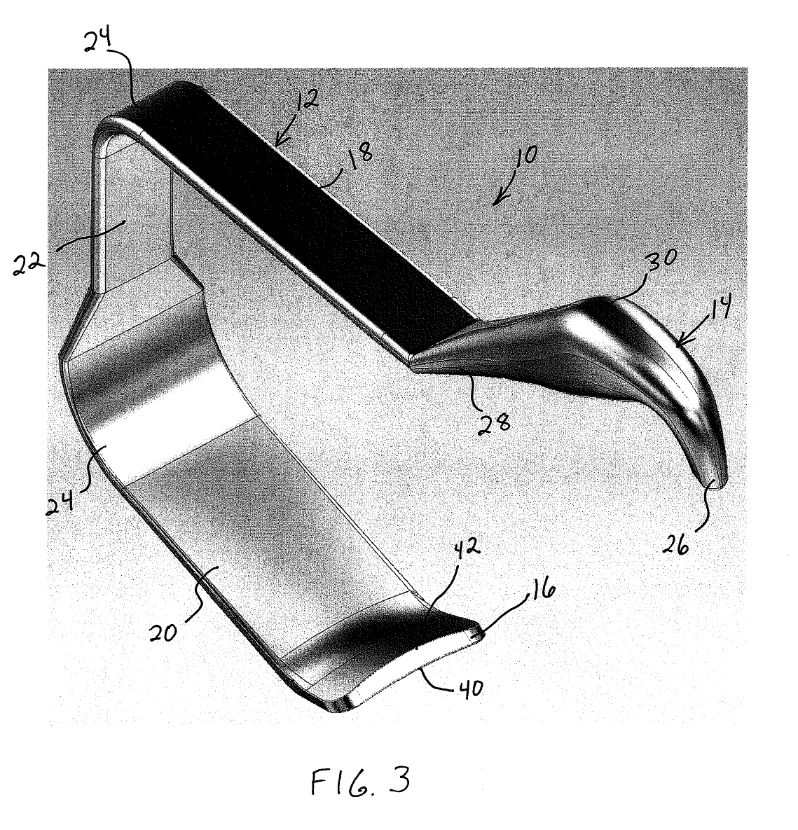

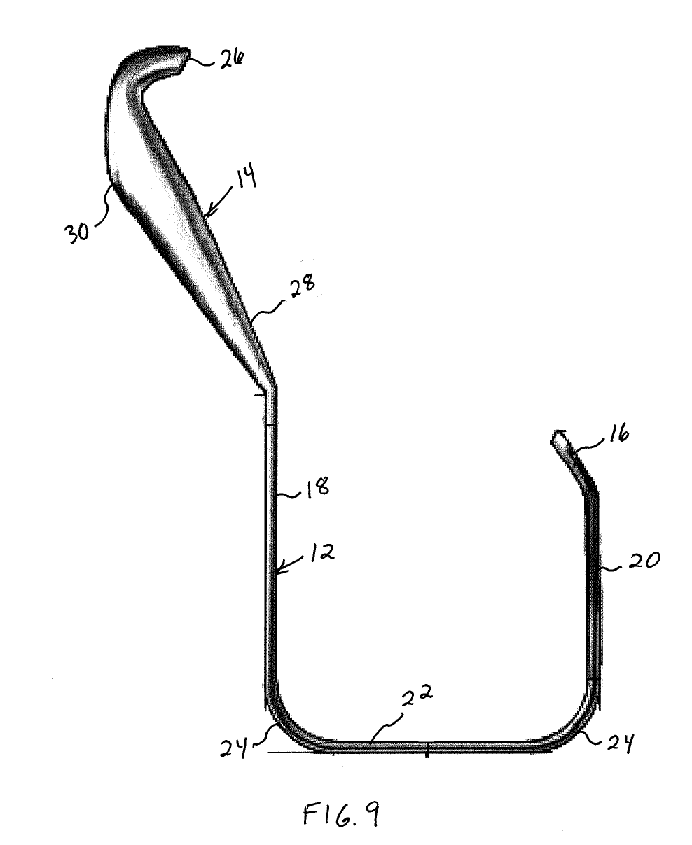

FIGS. 3 and 5b demonstrate that the angled member 16 may include a concave surface 40, an opposing convex surface 42, and a thickness extending therebetween. For instance, the radius of curvature of the concave 40 and convex 42 surfaces may be about 2.5-3 inches (e.g., about 2.8 inches) and about 5-6 inches (e.g., about 5.6 inches), respectively. However, the angled member 16 could have a substantially flat profile with opposing planar surfaces and may include a rectangular cross section in alternative embodiments. A respective width of the angled member 16 and the second portion 20 of the U-shaped member 12 may progressively increase along their respective lengths, as illustrated in FIGS. 6 and 7, although FIGS. 1-3 demonstrate that the angled member and the second portion may have approximately the same width if desired. For example, the second portion 20 may progressively increase in width at an angle “C” of about 5-10° (e.g., about 8°), while the angled member 16 may progressively increase in width at an angle “D” of about 25-35° (e.g., about 32°). The angled member 16 typically has a length less than the second portion 20 and may have a larger width than the second portion, as shown in FIGS. 4 and 6-8, which may facilitate insertion and placement of an implant by forming a pocket at the implant site. As shown in FIGS. 1-5 and 7-11 and mentioned briefly above, the angled member 16 extends at an oblique angle with respect to the longitudinal axis of the second portion 20 of the U-shaped member 12, which may push the tissue away from the desired implant site in order to form a pocket. Thus, the size and configuration of the angled member 16 facilitates forming a pocket at the site of implantation and holding the soft tissue up from the pocket, while the size and configuration of the second portion 20 forms a channel between a remote incision and a desired implant site, as explained in greater detail below.

The following description provides one exemplary technique for using a medical device 10 in a breast augmentation procedure and is not meant to be limiting. In use, an assistant (generally designated “A”) may position himself or herself on either side of an operating table for assisting a surgeon with the procedure. FIG. 12 illustrates that the assistant A is located on the side of the operating table opposite the site of the incision 34. For use with a transaxillary approach, an assistant A or surgeon would insert the device 10 through an incision 34 made by a surgeon in the patient's underarm 36 by first inserting the angled member 16 with the angled member oriented such that the end of the angled member is positioned upwards and towards the patient's skin. The assistant A or surgeon would then move the angled member 16 towards the breast and the site of implantation such that the second portion 20 is also inserted within the incision 34. Thus, at least a portion of the second portion 20 would be inserted within the incision 34 and into the patient's body in order to form a channel 38 between the incision and the site of implantation, while also holding open the incision. The second portion 20 would typically be oriented within the patient's body such that the planar surfaces of the second portion 20 are generally aligned with the patient's skin or a coronal plane. The angled member 16 is sized and configured to form a pocket at the implant site. Once the angled member 16 and second portion 20 are in position, the surgeon is able to insert a breast implant through the incision 34 and deliver the implant to the implant site. As known to those of ordinary skill in the art, the breast implant may be positioned within a subglandular, subpectoral, or submuscular pocket depending on a patient's and surgeon's preferences. While the assistant holds the device 10 in position, the surgeon may then manipulate the implant through the incision and work the implant through the channel 38 and into the pocket at the implant site. The surgeon may then position the implant within the pocket until the desired position and appearance are obtained. Different types of implants may be delivered using the aforementioned technique, although the medical device 10 is particularly useful for delivering a silicone implant.

Embodiments of the present invention may provide several advantages. According to one embodiment, the medical device 10 may facilitate insertion and placement of a breast implant to a desired implant site located remotely from the incision. For instance, the medical device 10 may be used with transaxillary incisions for implanting silicone implants, wherein silicone implants are typically not delivered using an incision site remote from the implant site. Moreover, the medical device 10 is easy to use, while the size and configuration of the U-shaped member 12 facilitates use by an assistant on either side of an operating table. In addition, the medical device 10 is sized and configured to hold the incision open, as well as provide a more lubricious surface for placement of the implant. Furthermore, the angled member 16 and the second portion 20 are of sufficient length to allow for continuity of the channel and the pocket at the implant site.

Many modifications and other embodiments of the invention set forth herein will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A medical device for use with a surgical procedure comprising:
a U-shaped member comprising a first portion having a first end, a second portion having a second end, and a third portion extending between the first and second portions opposite the first and second ends, the second portion having a longitudinal axis;
a handle extending from the first end; and
an angled member extending from the second end at an oblique angle with respect to the longitudinal axis of the second portion,
wherein the angled member and at least a portion of the second portion are configured to be placed through an insertion site in a body for facilitating a surgical procedure.

2. The medical device of claim 1, wherein the angled member and the second portion of the U-shaped member have approximately the same width.

3. The medical device of claim 2, wherein the angled member and the second portion of the U-shaped member each has a width of about 1-2 inches.

4. The medical device of claim 1, wherein a respective width of the angled member and the second portion of the U-shaped member progressively increases along their respective lengths.

5. The medical device of claim 1, wherein the first and third portions of the U-shaped member have approximately the same width and thickness.

6. The medical device of claim 5, wherein the first and third portions of the U-shaped member have a width of about 0.5-1 inch.

7. The medical device of claim 1, wherein a longitudinal axis of the first portion is non-parallel to the longitudinal axis of the second portion.

8. The medical device of claim 1, wherein a longitudinal axis of the first portion and the longitudinal axis of the second portion are each straight.

9. The medical device of claim 1, wherein the longitudinal axis of the first portion of the U-shaped member extends at an oblique angle with respect to a longitudinal axis of the third portion.

10. The medical device of claim 9, wherein the longitudinal axis of the first portion of the U-shaped member extends at an angle of about 95-115° with respect to the longitudinal axis of the third portion.

11. The medical device of claim 1, wherein a longitudinal axis of the first portion of the U-shaped member extends at an oblique angle with respect to the handle.

12. The medical device of claim 1, wherein the angled member extends at an angle of about 25-45° with respect to the longitudinal axis of the second portion of the U-shaped member.

13. The medical device of claim 1, wherein the second portion of the U-shaped member comprises a pair of opposing planar surfaces and a thickness extending therebetween.

14. The medical device of claim 1, wherein the angled member comprises a concave surface, an opposing convex surface, and a thickness extending therebetween.

15. The medical device of claim 1, wherein the angled member has a length of about 0.5-1 inch.

16. The medical device of claim 1, wherein the first portion of the U-shaped member has a length of about 4-5 inches.

17. The medical device of claim 1, wherein the second portion of the U-shaped member has a length of about 2.5-3.5 inches.

18. The medical device of claim 1, wherein the third portion of the U-shaped member has a length of about 4.5-5.5 inches.

19. The medical device of claim 1, wherein the U-shaped member, the handle, and the angled member are integrally formed from a single piece of solid material.

20. The medical device of claim 1, wherein a sum of interior angles defined between the first and third portions, the second and third portions, and the second portion and the angled member is less than about 360°.

21. The medical device of claim 1, wherein the angled member has a length less than the second portion.

22. The medical device of claim 1, wherein an aspect ratio of a width to a thickness of the first, second, and third portions is about 3-8.

23. The medical device of claim 1, wherein an aspect ratio of a width to a thickness of the angled member is about 3-10.

24. The medical device of claim 1, wherein the medical device is configured for use with a breast augmentation procedure for facilitating the insertion of a breast implant, and wherein the angled member and at least a portion of the second portion are configured to be placed through a remote insertion site for guiding a breast implant to a desired location.

25. A medical device for use with a surgical procedure comprising:
a U-shaped member comprising a first portion coupled to a second portion, the second portion having a longitudinal axis;
a handle extending from the first portion of the U-shaped member; and
an angled member extending from the second portion at an oblique angle with respect to the longitudinal axis of the second portion and having a length less than the second portion,
wherein the angled member and at least a portion of the second portion of the U-shaped member are configured to be placed through an insertion site in a body for facilitating a surgical procedure.

26. A method comprising:
providing a medical device comprising: a U-shaped member comprising a first portion coupled to a second portion, the second portion having a longitudinal axis; a handle extending from the first portion; and an angled member extending from the second portion at an oblique angle with respect to the longitudinal axis of the second portion;
forming an incision in a body;
inserting the angled member and at least a portion of the second portion of the U-shaped member within the incision such that the second portion of the U-shaped member extends from the incision to a desired location within the body and the angled member is positioned at the desired location; and
performing a surgical procedure while at least a portion of the second portion of the U-shaped member and the angled member are positioned within the body.

27. The method according to claim 26, wherein forming an incision comprises forming a transaxillary incision.

28. The method according to claim 26, wherein forming an incision comprises forming a transabdominal incision.

29. The method according to claim 26, wherein forming an incision comprises forming an incision in the body at a location remote from a desired implant site.

30. The method of claim 29, wherein inserting comprises inserting the angled member and at least a portion of the second portion of the U-shaped member within the incision such that the second portion of the U-shaped member extends from the incision to a desired implant site and the angled member is positioned at the desired implant site, and wherein performing comprises guiding a breast implant to the desired implant site while at least a portion of the second portion of the U-shaped member and the angled member are positioned within the body.

31. The method according to claim 30, wherein guiding comprises guiding a silicone breast implant to the desired implant site.

32. The method according to claim 30, wherein inserting comprises positioning the angled member such that the angled member defines a pocket at the desired implant site.

33. The method according to claim 30, wherein inserting comprises inserting the second portion of the U-shaped member such that the second portion forms a channel between the incision and the desired implant site.
